(19) United States
(12) Patent Application Publication
(10) Pub. No.: US 2025/0366545 A1
(43) Pub. Date: Dec. 4, 2025

(54) COMPRESSION TROUSERS

(71) Applicant: Julius Zorn GmbH, Aichach (DE)

(72) Inventors: Christian TAEGER, Munich (DE); Mathias LEITLOFF, Bindlach (DE)

(73) Assignee: Julius Zorn GmbH, Aichach (DE)

(21) Appl. No.: 19/218,743

(22) Filed: May 27, 2025

(30) Foreign Application Priority Data
May 29, 2024 (DE) 10 2024 115 127.7

(51) Int. Cl. A41D 1/06

(52) U.S. Cl. CPC A41D 1/06; A41D 2200/10; A41D 2300/32; A41D 2400/32

(57) ABSTRACT

Compression trousers for a compressive treatment of edema or other diseases or injuries in a genital or pelvic region of a patient. The compression trousers include a compression element for exerting a compression on the genital region and/or the pelvic region of the patient, a pants part, detachable fastening elements and at least one waistband which is non-detachably fastened to or molded onto the compression element. The at least one waistband is configured to be fastened in a waist region or in an upper hip region of the patient by the detachable fastening elements. The compression element is permanently fastened to the pants part by a non-detachable fastening member and the pants part is fully enclosing at least the pelvic region of the patient, when the compression trousers are put on a patient.

### FIELD OF THE INVENTION

The invention relates to compression trousers, in particular for the compressive treatment of edema or other diseases or injuries in the genital or pelvic region of a patient or for supporting postpartum recovery after childbirth.

### BACKGROUND

EP 2 884 862 B1 discloses a medical garment in the form of a hip and abdominal support for supporting the pelvis and abdomen of a patient to treat and control pain, edema and infection associated with acute or chronic conditions such as cancer or following genital or abdominal surgery or vaginal delivery to reduce complications and improve recovery time. The hip and abdominal support thereby comprises a waist encircling member adjustably attachable at its front portion and having an upper and a lower edge and a rear portion, and shorts non-removably attached to the lower edge of the waist encircling member and having a crotch portion and a strap member, wherein the strap member is attached at one end to the rear portion of the waist-encircling member, and the other end thereof, after passing under the crotch portion of the shorts, is fixable to the other end of the front of the waist-encircling member via a hook-and-loop fastener so that the strap member exerts pressure on the crotch portion of the wearer.

The disclosed hip and abdominal support is a one-piece and individually adjustable medical garment that can be worn over the underwear immediately after an operation or after childbirth and thereafter during a recovery period of several weeks of the person to be treated in order to support the back, the abdominal muscles and the vulva. The application of the medical garment, in particular the fastening of the strap elements by passing the strap elements under the crotch area, proves to be cumbersome and, depending on the type of impairment of the wearer, painful. The disclosed garment also proves to be disadvantageous in terms of flexibility and individual adaptation to different patients with different size ratios in the abdominal and hip area. Further disadvantages of the disclosed medical garment in terms of wearing comfort result from the full attachment of the shorts to the member surrounding the waist, for example when bending over, because the waist-encircling member is pulled downwards.

In other applications for medical hip and abdominal supports, such as the compressive treatment of genital edema or other edema in the pelvic or groin area, it is also advantageous if a predetermined compression pressure can be generated in the hip area. The disclosed hip and abdominal support is not suitable for this purpose. Furthermore, with the disclosed hip and abdominal support, it is hardly possible to individually adjust the compression pressure exerted to the patient's clinical picture or the healing process. In addition, the known hip and abdominal support proves to be uncomfortable when an increased compression pressure on the genital area is to be set with the three straps of the strap element, because the narrow straps can constrict the genitals. In particular, due to the narrow design of the individual strap elements, it is not possible to generate a flat and uniform compression pressure on the genital area with the disclosed hip and abdominal support. Correct adjustment of the strap elements proves to be difficult with the disclosed hip and abdominal support. Incorrectly positioned strap elements can have a significant impact on the wearing comfort and can also lead to uneven pressure distribution and cause pain.

### SUMMARY OF THE INVENTION

Against this background, an object of the invention is to provide compression trousers for the compressive treatment of edema or other diseases or injuries in the genital or pelvic region of a patient, which enable an individual and a precise adaptation to the shape of the patient's body, the setting of a desired, individually and precisely adaptable compression pressure both in the genital region and the groin area and in the hip region, and comfortable wearing over a longer treatment period. The compression pressure generated by the compression trousers should be easily and precisely adjustable during the treatment period with a precise adjustment of the desired compression pressure, and it should be adaptable to the success of the treatment.

The compression trousers disclosed in the following description can be used in particular for the compressive treatment of edema or other diseases or injuries in the genital or pelvic region of a patient and can be worn in particular by male and also by female patients, for example for postpartum recovery after childbirth. The disclosed compression trousers comprise a compression element for exerting compression on the genital region and/or the pelvic region of the patient, a pants part and at least one waistband, which is non-detachably fastened to or integrally formed on the compression element, wherein the at least one waistband can be put on the patient adjustably in the region of the waist or in the upper hip region of the patient by means of detachable fastening elements, wherein the pants part fully encloses at least the pelvic region of the patient when put on the patient. Therein, the compression element is non-detachably attached to the pants part.

In accordance with an aspect of the invention the compression trousers comprise
a compression element for exerting a compression on the genital region and/or the pelvic region of the patient,
a pants part,
at least one waistband which is permanently fastened to the compression element, and
detachable fastening elements attached on the waistband,
wherein the compression element is permanently fastened to the pants part by non-detachable fastening means,
wherein the pants part is fully enclosing at least the pelvic region of the patient, when the compression trousers are donned to a patient,
wherein the at least one waistband is configured to be fastened in a waist region or in an upper hip region of the patient at different positions along a cranial direction by the detachable fastening elements,
wherein the compression element is made of an elastic material, which is stretched when the compression trousers are put on the patient with the waistband being fixed in a selected position along the cranial direction by the detachable fastening elements,
wherein the compression element exerts a compression pressure on the genital region of the patient by the stretching of the elastic material when the compression trousers are put on the patient, and
wherein the compression pressure exerted on the genital region of the patient is adjustable by selecting the position of the waistband on the patient in the cranial direction.

A non-detachable fastening or attaching is considered to be equivalent to a permanent fastening or attaching and is understood here to mean that two parts that are non-detachably or permanently connected to each other (such as the waistband attached to the compression element and the compression element attached to the pants part) cannot be separated from each other, at least not without a tool or aid. Non-detachable or permanent connections in this sense can be produced in particular by seaming, bonding or welding or also by a one-piece or integral attachment, in particular by a one-piece material formation of the two parts, such as a one-piece (in particular one-piece material) molding of one part to the other part. In contrast, a detachable fastening means that the two detachably connected parts can be separated from each other manually and without the use of tools or aids. Detachable connections can be provided in particular by Velcro® (hook and loop) fasteners, adhesive fasteners, press stud fasteners or belt buckles.

The non-detachable attachment or permanent fastening of the compression element to the pants part results in one-piece compression trousers which allows the compression pressure to be adjusted more easily and quickly than the compression trousers with belt elements known from the prior art. In particular, the patient himself can easily adjust the compression pressure of the compression trousers disclosed herein by varying the position of the waistband in the waist area or in the upper hip area of the patient. The non-detachable or permanent attachment of the compression element to the pants part enables a simple, fast, targeted and precise adjustment of a defined compression pressure to be exerted by the compression element on the genital area or the groin area of the patient, whereby the compression pressure exerted on the genital area or the groin area of the patient depends on the position of the pants part and the waistband on the patient's body in a cranial direction (e.g. a direction along the longitudinal direction of the patient's body towards the patient's head).

In a preferred embodiment, the at least one waistband comprises first fastening means and the pants part comprises second fastening means arranged in a waistband region of the pants part, wherein the pants part can be detachably fastened to the waistband by means of a detachable coupling of the first fastening means and the second fastening means.

As explained above, a detachable coupling means that the two detachably connected parts (here, for example, the pants part and the waistband) can be separated from each other manually and without the use of tools or aids. Such detachable connections can be provided for example by Velcro® or adhesive fasteners or by button or press stud fasteners.

The detachable coupling of the first fastening means and the second fastening means for fastening the pants part to the waistband enables a targeted and a precise adjustment and a fixing of a defined compression pressure exerted by the compression element on the genital area or the groin area of the patient and prevents the pants part from slipping. The first fastening means and the second fastening means are suitably designed such that they can be coupled to one another at different positions along the cranial direction of the patient, with the compression pressure exerted on the patient in the genital area or the groin area of the patient depending on the cranial position at which the first and second fastening means are coupled to one another.

The first fastening means and the second fastening means are preferably designed as corresponding components of a two-part, reversible connecting means, in particular a hook-and-loop fastener, an adhesive fastener or a snap fastener

The first fastening means are designed, for example, as a flat fleece part of a Velcro® fastener, which is arranged on an outer side of the waistband. The second fastening means can be designed as a plurality of corresponding hook parts of a hook-and-loop fastener which are arranged on an inside of the pants part. As a result, the second fastening means (hook parts) can be fixed to the first fastening means (loop part) at a plurality of possible fixing positions in the cranial direction of the patient in order to set a desired compression pressure of the compression element and to maintain the compression pressure permanently during a treatment period or a cycle of a treatment period. The higher the cranial fixing position of the second fixing means (hook parts) on the first fixing means (fleece part), the more is the elastic compression element of the compression element stretched and the higher is the compression pressure exerted by the compression element on the genital area and the groin area of the patient.

The compression element is preferably designed as a one-piece bag or as a one-piece apron and is made of an elastic textile material. By stretching the compression element in the cranial direction, a uniform compression pressure is generated when the compression trousers are put on the patient. This results in an increased wearing comfort and a better and evenly distributed compression pressure, as compared to the compression trousers known from the prior art.

The compression trousers disclosed herein are thus particularly suitable for decongesting an edema in the best possible way as part of a complex physical decongestion therapy and thus is preparing for an application phase in which the accumulation of body fluid is avoided. The compression element, which is shaped as a bag or apron, is appropriately formed and dimensioned, so that the compression element at least largely or preferably completely encloses the patient's genital area. This ensures that a uniform compression pressure is exerted on the full extension of an oedema in the patient's genital area.

Due to the preferred pouch-shaped or apron-shaped design of the compression element, the compression element can have an anatomical fit adapted to the patient's body, which preferably fully encompasses an edema in the genital area of the patient. On the one hand, a pouch-shaped or apron-shaped design of the compression element improves the wearing comfort and enables a uniform, extensive compression pressure to be exerted on an oedema in the genital area of the patient.

In one embodiment of the compression trousers designed for men, the pouch-shaped compression element is adapted to the patient's anatomy in such a way that the men's genitals can be completely encompassed by the compression element. This enables the compression element to completely surround the edema to be treated. A bag-shaped compression element also can act as a support bag for the men's genitals.

The waistband can be detachably fastened in the area of the patient's waist or upper hip area by means of the fastening elements arranged on the waistband. The fastening elements can preferably be fixed in different positions in a circumferential direction of the patient. This makes it possible to adjust the waistband to the patient's individual size and to precisely set the pressure exerted by the waistband on the waist area and/or the hip area of the patient.

The waistband further is configured to be fastened in the area of the patient's waist or upper hip area at different positions in the cranial direction by means of the fastening elements. By fixing the waistband at different cranial positions, the compression pressure exerted by the compression element on the genital area of the patient can be adjusted to desired degrees of compressions. When fixing the waistband at a higher position in the cranial direction (e.g. towards the head of the patient), the elastic material of the compression element is more stretched and thus is producing a higher compression in the genital's region of the patient.

The fastening elements of the waistband are preferably designed as detachable fastening elements, in particular as a Velcro® fastener, hook fastener, snap fastener or belt buckle, and are preferably arranged in an end section of the waistband. This allows the waistband to be comfortably and securely fastened around the patient's waist or hip circumference with a slight stretching of the waistband. The fastening elements are preferably arranged in the patient's abdominal area, allowing the patient to comfortably put on and secure the waistband. By fixing the fastening elements at different positions in a circumferential direction of the patient, a pressure exerted by the waistband on the waist area and/or the hip area of the patient can be adjusted.

In a preferred embodiment of the compression trousers, the waistband comprises a first band section and a second band section, which are each non-detachably attached to the compression element and which can be connected to each other by means of the detachable fastening elements, in particular a Velcro® fastener or a belt buckle, and which can be adjusted, in particular variably, in the circumferential direction of the patient. A waistband consisting of two band sections makes the waistband easier to put on and the compression pressure exerted on the patient's abdomen can be adjusted more easily and more precisely. At the same time, the compression element permanently attached to the waistband can be prevented from slipping

In a preferred embodiment of the compression trousers, the compression element is attached to the pants part at a first end with a non-detachable connection, in particular with a first seam. The non-detachable connection is arranged on an inside of the pants part, in particular in a crotch area, which comes to lie in the crotch of the patient when the pants part is put on. The non-detachable connection of the compression element to the pants part in a crotch area ensures that the compression element fits correctly and develops a uniform, flat compression pressure in the correct area. The risk of an unfavorable pressure distribution due to an incorrect application of the compression trousers by the patient is minimized by the non-detachable attachment of the compression element to the pants part.

As explained above, a non-detachable fastening is understood that the two parts that are non-detachably connected to each other (here, for example, the compression element on the pants part) cannot be separated from each other, at least not without tools or aids.

The waistband of the compression trousers is attached to the compression element with a non-detachable connection, in particular with a second seam, so that different materials with properties tailored to the application can be used for the waistband and compression element. When the waistband is donned, the connection is located in particular in an abdominal area or pelvic area. In the embodiment with the waistband comprising two band sections, both band sections are attached to the compression element with non-detachable connections, in particular with second seams, whereby the connections are preferably arranged laterally on the compression element when the compression trousers are put on and at least essentially perpendicular to the circumferential direction (e.g. in cranial direction).

A non-detachable connection between the waistband and the compression element can also be designed by the waistband being molded onto the compression element. In the case of a waistband molded onto the compression element, the waistband and the compression element are made in one piece. It is also possible to form the waistband and the compression element from one material, for example from one piece of an elastic textile material, in particular from an elastic compression knit. The waistband can be formed seamlessly in one piece on the compression element, for example by seamlessly knitting the compression element to the waistband or by manufacturing the compression element and the waistband formed in one piece on it by cutting from a flat textile material. One advantage of a seamless or molded version of the waistband and the compression element is that it is more comfortable to wear, as local pressure and friction points due to seams on the inside of the compression trousers are avoided.

When the compression trousers are put on the patient, the compression element is preferably located inside the pants part. The compression element in particular is partially or completely covered by the pants part. This prevents pressure points due to a fold in the pants part arranged under the compression element. A pants part covering the compression element is also advantageous for aesthetic reasons.

The waistband is preferably also at least partially or completely covered by the pants part, when the compression trousers are put on the patient. A partial overlap of an upper section of the pants part, in particular a waist area of the pants part, with the waistband arranged underneath is advantageous, in particular with regard to a more comfortable wearing of the compression trousers. However, it is possible for the pants part to be arranged completely below and at a distance from the waistband on the patient and for the waist area of the pants part not to overlap the waistband. A waistband that protrudes at least slightly below the pants part is advantageous because it allows the waistband to be gripped manually, for example to enable to set, adjust or readjust the compression pressure exerted by the compression element by changing the coupling position of the fastening elements or by changing the position in which the waistband is applied on the patient in the cranial direction.

When the compression trousers are put on a patient, the compression element, which is made in particular from an elastic textile material, is stretched depending on the cranial position of the waistband and/or the cranial position of a waist area of the pants part. Due to the stretching of the elastic material of the compression element, the compression element produces a compression pressure on the genital area and the groin area of the patient, wherein the amount of this compression pressure is depending on the degree of stretching of the elastic material. Thus, the compression pressure exerted by the compression element in the on the genital area and the groin area of the patient can be adjusted by selecting the application positions of the waistband and the waist area of the pants part in the cranial direction and this compression pressure can also be changed after the compression trousers have been put on the patient, for example to adjust the compression pressure over the course of a longer treatment period. To adjust the compression pressure, it is only necessary to change the cranial position of the waistband and/or the waist area of the pants part, without having to remove the entire compression trousers.

Both the pants part and the compression element each are conveniently made from an elastic textile material. Preferably, the pants part is made of a compression knit or an elastic woven textile material. The compression element can likewise be made of a compression knit or a woven textile material. The waistband is preferably made of a foam laminated on both sides with a velour material. The elasticity of the waistband is preferably less than the elasticity of the pants part and the compression element.

The pants part of the compression trousers is preferably a compression textile made of an elastic textile material and is manufactured in particular from an elastic compression knit, the textile material preferably being selected and the pants part being dimensioned in such a way that the pants part exerts a predetermined compression pressure on the patient, in particular in the hip and pelvic region, when it is donned to the patient. It is particularly preferable for the pants part to be made from an elastic compression knit with a base knit and an elastic weft thread inserted into it. This makes it possible to set a desired compression pressure by selecting and stretching the weft yarn material and adapting the dimensions of the pants part to the patient's body shape and anatomy, in particular by an individualized manufacturing. The pants part can also be made from a circular knitted yardage of an elastic textile material.

The design of the pants part from a compression textile means that, in addition to the pressure exerted by the compression element on the patient's genital area, a fully effective compression pressure is also generated on the hip area and/or the pelvic area of the patient. This means that an edema extending from the genital area via the groin area to the hip and pelvic area can be effectively treated with a compression therapy by using the compression trousers.

In a further embodiment, the pants part can have an elastic waistband and/or a tunnel waistband in the waist area in order to enable an easy and lasting fixing of the (cranial) position in which the pants part is put on the patient. Furthermore, to improve the fit of the compression trousers on the patient, suspenders can be arranged on the pants part and/or on the waistband.

In a preferred embodiment, a pad is provided on the inside of the compression element, which faces the patient's body when the compression trousers are put on, whereby the pad can be inserted and detachably attached to the compression element, for example via a Velcro® fastener. The pad serves to increase the pressure exerted by the compression element on an edema and appropriately has a shape adapted to the body shape of the patient to be treated. In particular, special pads with a fit suitably designed for men or women can be used in the crotch or perineal area. In addition to or instead of fastening the pad to the inside of the compression element by means of a detachable fastening means, such as a Velcro® fastener, a pocket can also be provided on the compression element, in particular on its inside, into which the pad can be removably inserted.

The disclosed compression trousers therefore enable an overall, individual and precise adjustment of a compression pressure exerted by the compression trousers on a genital edema or on the lower abdomen and hip area of the patient, as well as an improved wearing comfort. The waistband connected to the pants part via the compression element creates a one-piece pair of compression trousers that allows the compression pressure to be adjusted quickly and easily in order to treat a genital edema effectively. At the same time, the disclosed compression trousers ensure a high degree of flexibility in the adjustment of the compression trousers for different body shapes and circumferences of a patient to be treated.

### BRIEF DESCRIPTION OF THE DRAWINGS

These and other advantages and preferred features of the invention will become apparent from the description of an embodiment example described below with reference to the drawings. The drawings show in

FIG. 1: A detailed view of the inside of compression trousers with a waistband consisting of two band sections, a pants part and a compression element located inside the pants part and fastened in the crotch area of the pants part, wherein the inside of the compression element located in the pants part is shown;

FIG. 2: A front view of the compression trousers of FIG. 1 in a state applied to a patient;

FIG. 3: A rear view of the compression trousers of FIG. 2 in a state applied to the patient;

FIG. 4: A front view of the compression trousers of FIG. 1 with the pants part pulled down to better show the compression element and the waistband of the compression trousers, with the waist area of the pants part turned inside out;

FIG. 5: A rear view of the compression trousers of FIG. 4 with the pants part pulled down and with the waist area of the pants part turned inside out;

### DETAILED DESCRIPTION OF AN EMBODIMENT OF THE INVENTION

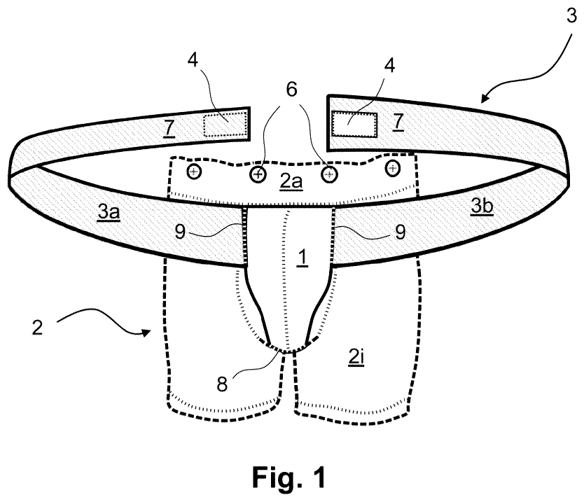

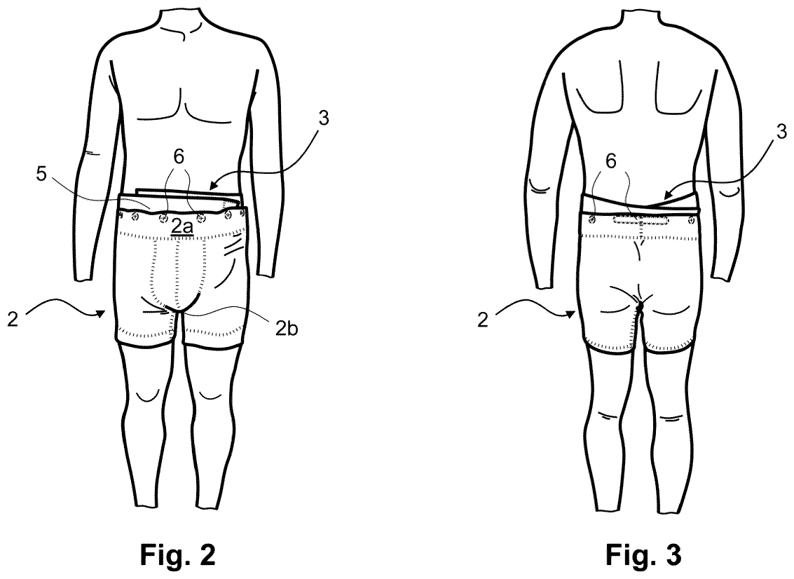

The embodiment of compression trousers shown in FIG. 1, which can be used in particular for the compressive treatment of edema or other diseases or injuries in the genital or pelvic region of a patient, comprises a waistband 3 with band sections 3a and 3b, a compression element 1 which is non-detachably attached to the band sections 3a, 3b and a pants part 2 which is non-detachably attached to the compression element 1. The illustration in FIG. 1 shows the turned-up compression trousers in an interior view, so that the inner sides 2i of the pants part 2 and the compression element 1 are shown. The dashed representation of the pants part 2 makes it clear that the compression element 1 and areas of the band sections 3a and 3b of the waistband 3 are covered by the pants part 2, when the compression trousers are put on the patient. In the donned state of the illustrated embodiment of the compression trousers, which is shown in particular in FIG. 2 and FIG. 3, the compression element 1 and the waistband 3 lie inside the pants part 2 and are completely or at least partially covered by it.

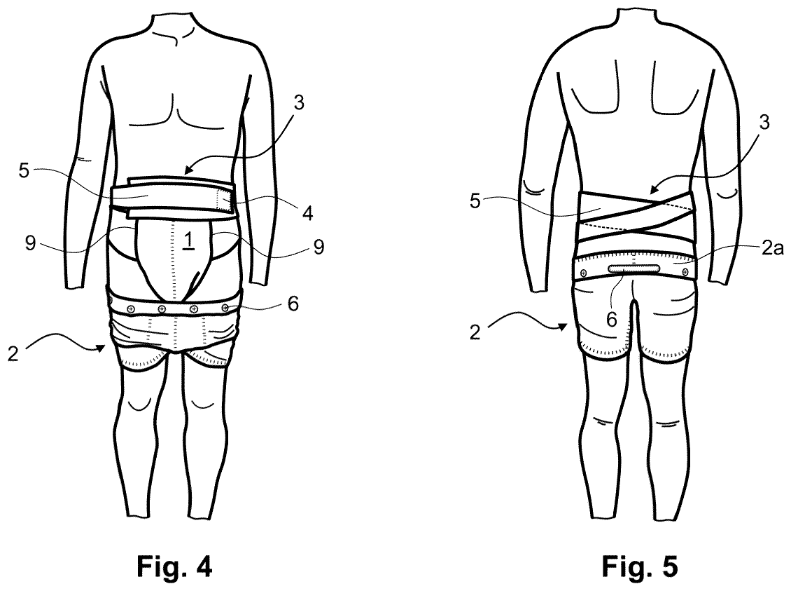

In the embodiment shown, the waistband 3 consists of a first band section 3a and a second band section 3b, each of which is non-detachably attached to the compression element 1 at one end with a second seam 9. When the waistband 3 is in place on the patient, the second seams 9 are located in particular in an abdominal area or a pelvic area of the patient. The outer side of the waistband 3, which can also be seen in FIG. 4 and FIG. 5, is designed as a flat fleece part of a Velcro® fastener, which forms the first fastening means 5. The first fastening means 5 is thus providing a large area for a coupling with second fastening means.

In an end section 7 at the free end of the band sections 3a and 3b, detachable fastening elements 4 are arranged, by means of which the band sections 3a and 3b can be connected to each other and, in particular, can be variably adjusted in the circumferential direction of the patient. The band sections 3a, 3b can also be looped several times around the patient's circumference. The detachable fastening elements 4 can be, for example, a Velcro® element with a hook-and-loop fastener, a hook fastener or a belt buckle. The waistband 3 can be fixed in the area of the patient's waist by means of the fastening elements 4 by coupling the fastening elements 4, for example by closing a belt buckle or by connecting the hook part of the Velcro® element to a loop part formed by the outside of the waistband 3. The fixation of the waistband 3 can be seen, for example, in the illustration in FIG. 4. By positioning the point of fixation of the fastening elements 4, a defined compression pressure exerted by the waistband 3 on the patient's waist can be adjusted. To remove the waistband 3 from the patient, the fastening elements 4 can be released, for example by releasing the coupling of the Velcro® fastener or opening the belt buckle.

In the embodiment shown, the compression element 1 is attached with the second seams 9 to the ends of the band sections 3a and 3b of the waistband 3 and comes to lie in the genital area and/or the pelvic area of the patient when the compression trousers are put on, which is particularly evident from FIG. 4. The second seams 9 are arranged on the lateral edges of the compression element 1 in an upper area and come to lie, in particular, in an abdominal area or a pelvic area of the patient, when the compression trousers are put on the patient. The compression element 1 is designed as a one-piece pouch or as a one-piece apron and is made of an elastic textile material.

The compression element 1 is attached to the pants part 2 at a first end with a non-detachable connection, in particular with a first seam 8 (FIG. 1). In the embodiment shown in the drawings, the first seam 8 connects a first end of the compression element 1 to a crotch area 2b on an inner side of the pants part 2. When the compression trousers are put on, the first seam 8 comes to rest in the patient's crotch.

FIG. 2 and FIG. 3 show the compression trousers in a front view and a rear view in a state applied to a patient. The pants part 2 of the compression trousers is located on the outside and in partial overlap with the waistband 3 underneath. In the embodiment example shown in the drawing, the pants part 2 is designed as a short with a tubular torso section, which contains a crotch area 2b in the middle, two tubular leg sections arranged on the torso section and a waist area 2a arranged on the upper edge of the torso section. The length of the two leg sections is variable, i.e. the length of the leg sections can also be longer or shorter than in the embodiment example shown here in the drawing. In particular, the pants part 2 can also be designed as tights with leg sections, whereby the leg sections can extend up to the patient's ankle and can optionally also include a foot section.

In the embodiment example shown in the drawing, detachable second fastening means 6, in particular in the form of a plurality of hook parts of a hook-and-loop fastener or press studs, are provided on the inside 2i of the pants part 2 in the waist region 2a, as can be seen in particular from FIG. 1, FIG. 4 and FIG. 5. FIG. 4 and FIG. 5 show a front view and a rear view of the compression trousers in a state in which it is put on the patient, wherein the compression element 1 and the waistband 3 are made visible by a partially pulled-down pants part 2 and the waist region 2a of the pants part being folded over outwards. The second fastening means 6 serve to detachably fasten the pants part 2 to the first fastening means 5, which are provided on an outer side of the waistband 3. Preferably, the second fastening means 6 are designed as a hook-and-loop tape with hooks, which interact with the hook-and-loop pad (loop part) of a hook-and-loop fastener arranged on the outside of the waistband 3. By coupling the second fastening means 6 of the pants part 2 with the first fastening means 5 of the waistband 3, the pants part 2 can be detachably fixed at selected positions on the waistband 3. The fixing of the pants part 2 to the waistband 3 can be seen, for example, in the illustrations in FIG. 2 and FIG. 3 in a donned state.

To put the compression trousers on a patient, the patient first puts on the pants part 2 with the compression element 1 on the inside of the pants part 2 and the waistband 3 being opened, whereby the compression element 1 sewn onto the crotch area 2b of the pants part 2 comes to rest in the patient's crotch. As shown in FIG. 4 and FIG. 5, the waist area 2a of the pants part 2 initially remains below the patient's hip area and can be folded outwards.

In this state, the waistband 3 is placed around the patient's waist and fixed in place using the detachable fastening elements 4. By selecting the locking position of the fastening elements 4, a certain amount of pressure is exerted by the waistband 3 on the patient's waist and hip area.

After the waistband 3 has been put on, the pants part 2 is pulled upwards so that the compression element 1 is located inside the pants part 2 and is at least substantially completely covered by the pants part 2. When the pants part 2 is pulled upwards, the position of the waist area 2a of the pants part 2 can be adjusted to selected positions. In embodiments in which the compression trousers are comprising suspenders or an elastic waistband or a tunnel waistband arranged on the pants part 2, a position of the waist area 2a of the pants part above or below the waistband 3 can be selected.

Due to the elastic properties of the compression element 1, which is made of an elastic textile material, the compression element 1 is stretched more or less depending on the cranial position of the waistband 3 and the waist area 2a of the pants part. The degree of stretching of the compression element 1 and thus the compression pressure exerted by the compression element 1 on the genital area and the groin area of the patient can be adjusted or varied by selecting the cranial position of application of the of the waistband 3 and the waist area 2a of the pants part.

In the embodiment shown in the drawings, the pants part 2 can be fixed to the waistband 3 by coupling the second fastening means 6 arranged in the waist area 2a of the pants part 2 with the first fastening means 5 on the waistband 3. The compression pressure exerted by the compression element 1 on the genital area and the groin area of the patient thereby can be firmly adjusted via a selection of the fixing position of the first and second fastening means 5 and 6 in the cranial direction. The cranial fixing position, at which the second fastening means 6 are coupled to the flat first fastening means 5, thus determines the amount of compression pressure exerted by the compression element 1 on the patient's genital area.

In the preferred embodiment shown in the drawings, a plurality of hook parts of a hook-and-loop fastener or press studs are provided as second fastening means 6, which are arranged on the inside 2i of the pants part 2 in the region of the waistband 2a. With these second fastening means 6, the inner side 2i of the pants part 2, in particular in the area of the waistband 2a, can be removably fastened to the outer side of the waistband 3 lying underneath in the overlap area. If the second fastening means 6 are designed as press studs, the outer side of the waistband 3, for example, contains a plurality of corresponding receptacles with recesses corresponding to the number of press studs, into each of which a head part of a press stud arranged on the inner side of the waistband 2a of the pants part 2 can be releasably anchored, or vice versa.

To remove the compression trousers from the patient, the procedure described above for putting on the compression trousers on a patient is carried out in reversed order. For this purpose, the coupling of the first and second fastening means 5 and 6 is first released and then the pants part 2 is pushed downwards, as shown in FIG. 4 and FIG. 5. The detachable fastening elements 4 of the waistband 3 are then opened and the compression trousers can be removed from the patient.

The one-piece design of the compression trousers according to the disclosure enables the compression trousers to be put on and taken off easily and comfortably, and the compression pressure on the genital area and the groin area of the patient can be adjusted quickly and precisely by adjusting the stretch of the compression element 1 over an appropriate selection of the cranial application positions of the waistband 3 and the waist area 2a of the pants part 2. The compression pressure exerted by the compression element 1 can be readjusted quickly and easily afterwards, i.e. after the compression trousers have been put on the patient. This is advantageous, for example, in the case of a longer-lasting compression therapy, in order to be able to adapt the compression pressure to the healing process. For example, it is possible to gradually reduce the compression pressure during the course of a compression treatment, depending on the healing process.

Both the pants part 2 and the compression element 1 are each conveniently made from an elastic textile material. Preferably, the pants part 2 is made from a compression knit with a basic knit made from an elastic thread and an elastic weft thread inserted or knitted into it. The pants part 2 and the compression element 1 can also be made from a circular knitted fabric. In particular, the material of the pants part 2 can be a circular knitted fabric made of 80% polyamide and 20% elastane. The compression element 1 can likewise be made from a compression knit. The compression element 1 is preferably a woven textile material, for example made of 71% polyamide and 31% elastane. The waistband 3 is preferably made of a foam material laminated on both sides with a velour material, for example 74% polyamide, 11% polyurethane and 15% elastane. The stretchability of the waistband 3 is preferably less than the stretchability of the pants part 2 and the compression element 1.

1. Compression trousers for a compressive treatment of edema or other diseases or injuries in a genital or pelvic region of a patient, the compression trousers comprising:
a compression element for exerting a compression on the genital region and/or the pelvic region of the patient,
a pants part,
detachable fastening elements and
at least one waistband which is non-detachably fastened to or molded onto the compression element,
wherein the at least one waistband is configured to be fastened in a waist region or in an upper hip region of the patient by the detachable fastening elements, and
wherein the compression element is permanently fastened to the pants part by non-detachable fastening means and the pants part is fully enclosing at least the pelvic region of the patient, when the compression trousers are donned to a patient.

2. The compression trousers according to claim 1, wherein the at least one waistband comprises first fastening means and the pants part comprises second fastening means located in a waistband region of the pants part, wherein the pants part can be detachably fastened to the at least one waistband by means of a detachable coupling of the first fastening means and the second fastening means.

3. The compression trousers according to claim 2, wherein the first fastening means and the second fastening means are corresponding components of a two-part, reversible connecting means, the reversible connecting means comprising at least one of a hook-and-loop fastener, an adhesive fastener and a snap fastener.

4. The compression trousers according to claim 2, wherein the first fastening means is comprising a flat loop part of a hook-and-loop fastener and is arranged on an outer side of the waistband, and the second fastening means is comprising a plurality of hook parts of a hook-and-loop fastener and is arranged on an inner side of the pants part.

5. The compression trousers according to claim 1, wherein the compression element is formed as a one-piece bag or as a one-piece apron made of an elastic textile material.

6. The compression trousers according to claim 1, wherein the detachable fastening elements are configured to be fixed in different positions in a circumferential direction of the patient, wherein, when the detachable fastening elements are fastened in a selected position, a pressure is exerted by the waistband on the waist region and/or the hip region of the patient, wherein the amount of pressure exerted by the waistband on the waist region and/or the hip region of the patient depends on the selected position of the detachable fastening elements.

7. The compression trousers according to claim 1, wherein the waistband comprises a first band section and a second band section, wherein the first band section and the second band section are permanently fastened to the compression element, wherein the first band section and the second band section are configured to be connected to one another by means of the detachable fastening elements.

8. The compression trousers according to claim 1, wherein the detachable fastening elements are designed as a hook-and-loop fastener or as a hook fastener, wherein the detachable fastening elements are arranged in an end section of the waistband.

9. The compression trousers according to claim 1, wherein the compression element is permanently fastened to the pants part with an inseparable connection, wherein the inseparable connection is arranged in a crotch region, which is located in a crotch of the patient when the compression trousers are worn.

10. The compression trousers according to claim 1, wherein the at least one waistband is fastened to the compression element with an inseparable connection, wherein the inseparable connection is located in an abdominal region or a pelvic region of the patient when the waistband is put on.

11. The compression trousers according to claim 1, wherein the compression element is located inside the pants part, when the compression trousers are put on the patient, and wherein the pants part covers partly or completely at least one of the compression element and the waistband.

12. The compression trousers according to claim 1, wherein the compression element is made of an elastic material, the compression element is attached to the waistband and the pants part under stretching of the elastic material when the compression trousers are put on the patient, wherein the compression element exerts a compression pressure on the genital region of the patient when the compression trousers are put on the patient, and wherein the compression pressure is adjustable by selecting a position of application of at least one of the waistband and a waist area of the pants part on the patient.

13. The compression trousers according to claim, 1 wherein the pants part is designed as a compression textile made of an elastic textile material, wherein the elastic textile material is stretched when the compression trousers are put on the patient and exerts a predetermined compression pressure on the patient in the hip region and the pelvic region of the patient.

14. The compression trousers according to claim 1, wherein the pants part is comprising at least one of an elastic waistband and a tunnel waistband in a waist area of the pants part.

15. The compression trousers according to claim 1, further comprising suspenders, which are arranged on the pants part or the waistband.

16. The compression trousers according to claim 1, further comprising a removable pad which either can be detachably attached to the compression element or can be inserted into a pocket formed on the compression element.

17. Compression trousers for a compressive treatment of edema or other diseases or injuries in a genital or pelvic region of a patient, the compression trousers comprising
a compression element for exerting a compression on the genital region and/or the pelvic region of the patient,
a pants part,
at least one waistband which is permanently fastened to the compression element, and
detachable fastening elements attached on the waistband,
wherein the compression element is permanently fastened to the pants part by non-detachable fastening means,
wherein the pants part is fully enclosing at least the pelvic region of the patient, when the compression trousers are donned to a patient,
wherein the at least one waistband is configured to be fastened in a waist region or in an upper hip region of the patient at different positions along a cranial direction by the detachable fastening elements,
wherein the compression element is made of an elastic material, which is stretched when the compression trousers are put on the patient with the waistband being fixed in a selected position along the cranial direction by the detachable fastening elements,
wherein the compression element exerts a compression pressure on the genital region of the patient by the stretching of the elastic material when the compression trousers are put on the patient, and
wherein the compression pressure exerted on the genital region of the patient is adjustable by selecting the position of the waistband on the patient in the cranial direction.

18. The compression trousers according to claim 17, wherein the detachable fastening elements are configured to be fixed in different positions in a circumferential direction of the patient, wherein, when the detachable fastening elements are fastened in a selected position, a pressure is exerted by the waistband on the waist region and/or the hip region of the patient, wherein the amount of pressure exerted by the waistband on the waist region and/or the hip region of the patient depends on the selected position of the detachable fastening elements.

19. The compression trousers according to claim 17, wherein the compression element is located inside the pants part, and wherein the pants part covers the compression element when the compression trousers are put on the patient.
